(19) United States
(12) Patent Application Publication
(10) Pub. No.: US 2009/0118619 A1
(43) Pub. Date: May 7, 2009

(54) ULTRASONIC DIAGNOSTIC APPARATUS AND ULTRASONIC DIAGNOSTIC METHOD

(76) Inventor: Mitsuhiro Oshiki, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/280,357

(22) PCT Filed: Feb. 21, 2007
(86) PCT No.: PCT/JP2007/053149

§ 371 Date: Aug. 22, 2008

(30) Foreign Application Priority Data
Feb. 23, 2006 (JP) 2006-046080
Oct. 3, 2006 (JP) 2006-271489

(51) Int. Cl. A61B 8/14

(52) U.S. Cl. 600/459

(57) ABSTRACT

Provided are an ultrasonic diagnostic apparatus and method capable of obtaining an image having a preferable SN and no blur in a wide range from a shallow portion to a deep portion of the image without using a delay control for each transducer.

The method includes: a transmission/reception step for transmitting and receiving an ultrasonic wave by focusing to each of a plurality of focus points; an image acquisition step for acquiring an ultrasonic image by using the reception signals from the respective focus points; and a step for selecting a plurality of transducers for performing transmission and reception with the same transmission/reception frequency from a plurality of transducers for each of the focus points and forming a group of transducers.

The transmission/reception step transmits/receives ultrasonic waves by differentiating the transmission/reception phase of at least two transducers belonging to the same group corresponding to the position of each focus point.

### TECHNICAL FIELD

The present invention relates to an ultrasonic diagnostic apparatus for obtaining an ultrasonic image of a diagnostic region in an object to be examined in real time by scanning ultrasonic beams, particularly to a technique for obtaining an image having a preferable SN and no blur in a wide range of the image.

### BACKGROUND OF THE INVENTION

An ultrasonic diagnostic apparatus scans ultrasonic beams in many directions whose ultrasonic probe wherein a plurality of transducers are arranged has apertures for transmitting/receiving ultrasonic waves.

Generally, since ultrasonic waves have a tendency of being attenuated inside of the object's body, all kinds of efforts are made to provide ultrasonic diagnostic apparatuses capable of penetrating ultrasonic waves to a deep portion of the object's body as well as receiving the reflected ultrasonic waves from the deep portion thereof, so as to obtain images having preferable SN in deep portion that is having good penetration.

As an example, Patent Document 1 discloses the technique for obtaining an image by changing the transmitting focus point for each scan frame and performing correlation processing among frame data having different focus points, in order to obtain an image having uniform distance resolution over a plurality of visual field depths without lowering the frame rate of the image.

Patent Document 1: JP-A-2006-130009

### DISCLOSURE OF THE INVENTION

### Problems to be Solved

The technique in Patent Document 1 performs a delay control for each transducer in order to focus on a desired focus point. Also, the focus point for obtaining one frame data is only one. Therefore, this technique requires installation of delay circuit or function for delay processing in order to execute a delay control, whereby causing the device configuration to be complex and raising the cost for manufacturing. Also, in each frame data, distance resolution improves only in the vicinity of the focus points, and blurring will occur in the region apart from the focus points. Therefore, even though the transmitting focus point is changed for every scan frame and correlation processing is performed among the frame data, the correlation will be performed between the data of improved distance resolution and the blurred data in the respective focus points. As a result, in such obtained images, it is considered that the unsolved problem remains that blurring of the images over a plurality of visual field depths cannot be fully eliminated.

Given this factor, the objective of the present invention is to provide an ultrasonic diagnostic apparatus capable of obtaining an image having preferable SN with no blur over a wide range from shallow portion to deep portion of the image without using a delay control for each transducer.

### Means for Solving the Problem

The ultrasonic diagnostic apparatus of the present invention for solving the above-described problems comprises:

an ultrasonic probe having a plurality of transducers for transmitting/receiving ultrasonic waves;

transmitting/receiving means for transmitting/receiving ultrasonic waves by focusing them to each of the plurality of focus points;

image acquiring means for acquiring an ultrasonic image using the receiving signals from the respective focus points; and

transducer group forming means for forming a group of the transducers by selecting, from the plurality of transducers, a plurality of transducers for transmitting/receiving ultrasonic waves using the same transmitting/receiving frequency with respect to every focus point,

wherein the transmitting/receiving means transmits/receives ultrasonic waves by differentiating transmitting/receiving phases of at least two transducers which belong to the same group, corresponding to the position of the respective focus points.

Also, the ultrasonic diagnostic method of the present invention for solving the above-described objectives is configured having:

a transmitting/receiving step for transmitting/receiving ultrasonic waves by focusing the ultrasonic waves to the respective plurality of focus points;

an image acquiring step for acquiring an ultrasonic image using the receiving signals from the respective focus points; and

a step for selecting, from a plurality of transducers, a plurality of transducers having the same transmitting/receiving frequency with respect to every focus point to form a group of the transducers,

wherein the transmitting/receiving step transmits/receives ultrasonic waves by differentiating the transmitting/receiving phases of at least two transducers which belong to the same group, corresponding to the position of the respective focus points.

### EFFECT OF THE INVENTION

By the effect of the above-described configuration, the ultrasonic diagnostic apparatus and the ultrasonic diagnostic method of the present invention is capable of forming ultrasonic beams focused to the desired focus points by differentiating transmitting/receiving phases of at least two transducers which belong to the same group. Since the position of the focus points can be changed without using a delay control for each transducer due to such effect, it is possible to acquire an image having preferable SN and no blur in a wide rage from shallow portion to deep portion of the image.

### BEST MODE FOR CARRYING OUT THE INVENTION

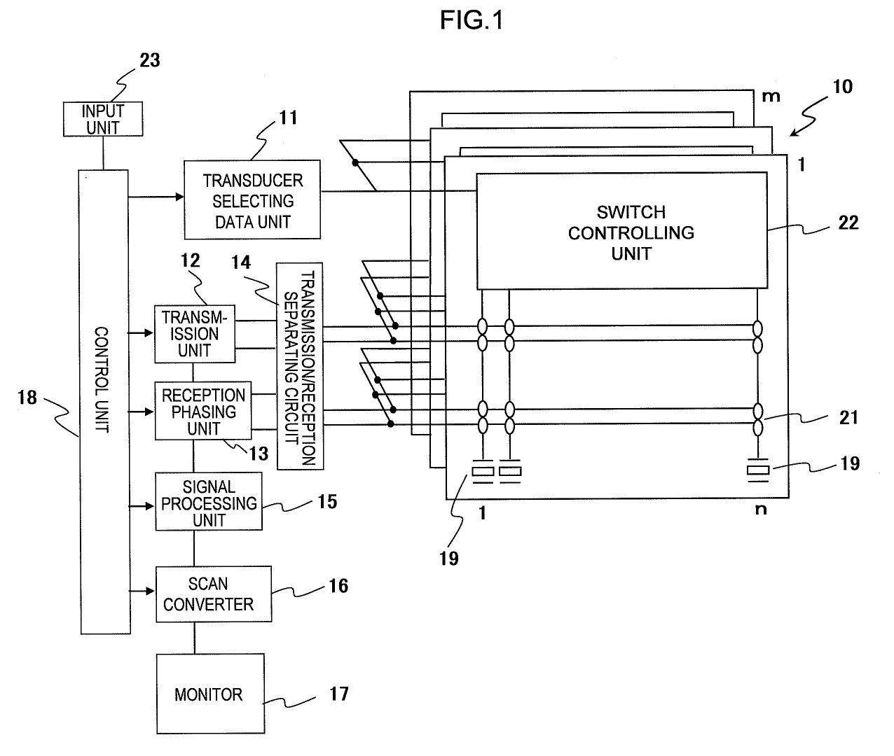

The respective embodiments of the ultrasonic diagnostic apparatus and the ultrasonic diagnostic method of the present invention will be described below on the basis of the diagrams. First, a general configuration of the ultrasonic diagnostic apparatus related to the present invention will be described using FIG. 1. FIG. 1 is a block diagram showing the general configuration of the ultrasonic diagnostic apparatus of the present invention.

The ultrasonic diagnostic apparatus of the present invention is configured by:

an ultrasonic probe 10 comprising a plurality of transducers;

a transducer selecting data unit 11 for selecting the transducers of the ultrasonic probe 10;

a transmitting/receiving unit 12 for transmitting transmission signals to the ultrasonic probe 10;

a reception phase unit 13 for phasing the receiving signals received by the ultrasonic probe 10;

a transmission/reception separating circuit 14 for switching the flow of the signals among the ultrasonic probe 10, transmitting/receiving unit 12 and reception phasing unit 13;

a signal processing unit 15 for processing the signals from the reception phasing unit 13;

a scan converter 16 for performing scan conversion of the ultrasonic scan and display scan with respect to the signals from the signal processing unit 15;

a monitor 17 for displaying the image data from the scan converter 16;

a control unit 18 for controlling the respective components; and

an input unit 23 for inputting the control signals to the control unit 18.

The ultrasonic probe 10 is a probe having one-dimensional or 2-dimensional array transducers 19. In particular, 2-dimensional array transducers have a configuration that m-number and n-number, that is m×n number of the transducers 19 are 2-dimensionally arranged in each direction of two orthogonal directions. The transmission/reception of ultrasonic waves is performed by a plurality of transducers being selected from the 2-dimensionally arrayed transducers 19 by the switching circuit 21 for each transmission/reception cycle of the ultrasonic beams. The 2-dimensional ultrasonic probe has a configuration that these 2-dimensional array transducers 19 and a switching circuit 21 are contained in a case. While the present invention will be described mainly exemplifying the 2-dimensional array transducers, it can be applied also to one-dimensional transducers.

A switch control unit 22 is for providing the signals for selecting the transducers to the switching circuit 21.

The transmitting unit 12 is configured having a digital memory storing transmitting waveform of a plurality of frequencies, a pulse generating circuit and an amplifying circuit. It is for providing driving signals to the transducers for transmitting ultrasonic waves to the inside of the object's body. It generates transmitting waves by the pulse generating circuit based on the transmitting waveform stored in the digital memory, amplifies them by the amplifying circuit and provides the driving signals to the probe via the transmission/reception separating circuit 14. The transmission/reception separating circuit 14 is for changing the passing direction of the signals upon transmission and reception.

The reflected waves (echoes) reflected in the body of the object are detected for each transducer, converted into electric signals (receiving signals) and amplified by an amplifier (not shown in the diagram) respectively.

The reception phasing unit 13 receives the plurality of receiving signals amplified by the amplifier and outputs the plurality of the signals by performing phasing addition so as to make them ultrasonic beam signals from a predetermined direction.

The signal processing unit 15 is for performing processing such as detection, logarithmic conversion, filtering and y compensation as preprocessing for imaging the receiving signals outputted from the reception phasing unit 13.

Scan converter 16 receives the receiving signals outputted from the signal processing unit 15 for each transmission/reception of the ultrasonic beam, digitalizes and accumulates the signals and forms image data. Then it outputs the accumulated image data in accordance with the scanning of the image display device. In other words, the scan converter 16 performs the scan conversion of the ultrasonic scan and the display scan.

The monitor 17 is a display that converts the image data outputted from the scan converter 16 into luminance signals and displays them as an image.

The control unit 18 controls the above-mentioned respective units directly or indirectly in accordance with the scan parameter inputted from the input unit 23 so as to cause the apparatus to transmit/receive ultrasonic waves and to display images. The control unit 18 is, for example, a central operation unit (CPU).

Next, operation of the above-mentioned ultrasonic diagnostic apparatus will be described. The ultrasonic diagnostic apparatus comprising the ultrasonic probe 10 having 2-dimensional array transducers related to the present invention uses, for example, Fresnel binding focus technique disclosed in Patent Document 2. That is the technique to select, from the 2-dimensional array transducers, the transducers included in the same distance range from a predetermined point (for example, the center) and binds them in a circular or ring-shaped concentric circle. The concrete description is as follows.

The operator applies the ultrasonic probe 10 to a body surface of a diagnostic region in an object to be examined, inputs a scan parameter such as transmitting focus depth from the input unit 23, and inputs the command to start ultrasonic scanning. Corresponding to this command, the control unit 18 controls the respective units and starts ultrasonic scanning. First, the control unit 18 outputs the commands, with respect to the switch control unit 22 and the transmitting unit 12, to select the transducers for the first transmission, and to set the transmitting waveform corresponding to the drive pulse outputting command and the transmitting focus depth. When these commands are executed, the driving pulses are provided to the ultrasonic probe 10 from the transmitting unit 12.

The switching circuit 21 in the ultrasonic probe 10 connects the transmitting unit 12 and a driving pulse inputting line of the transducer so as to form a circular or ring-shaped transducer bundle to be described later or a group of those transducer bundles, according to the command of the controlling unit 18 corresponding to the transmitting focus depth inputted by the operator from the input unit 23. When the driving pulses are inputted, the respective transducers oscillate at predetermined frequency and transmit ultrasonic waves to the inside of the object's body.

Patent Document 2: JP-2000-325344

As for the ultrasonic waves transmitted to the inside of the object's body, part of them are reflected at a plane having different acoustic impedance of the tissues or organs in the living body and returned in the direction of the ultrasonic probe 10 as echoes. The control unit 18 controls the reception system in order to receive these echoes. First, upon completion of transmission, the control unit 18 performs switching selection for connecting the transducers for receiving and the reception phasing unit 13 to the switch circuit 21. Also upon reception, the control unit 18 performs the switching selection of transducers so as to form a circular or ring-shaped transducer bundle or a group of the transducer bundles thereof in the same manner as upon transmission. The details thereof will be described later.

The receiving signals received in each group of transducer bundles are phased and added in the reception phasing unit 13 and outputted to the signal processing unit 15 as the receiving signals formed as a beam. The signal processing unit 15 performs the previously mentioned processing with respect to the inputted receiving signals, and outputs the signals after processing to the scan converter 16. The scan converter 16 stores the inputted signals to the memory, reads out and outputs the stored content corresponding to the synchronized signals for displaying to the monitor 17. When the above operation is completed, the control unit 18 repeats the same series of operations by sequentially changing the position or direction of transmission/reception of ultrasonic waves.

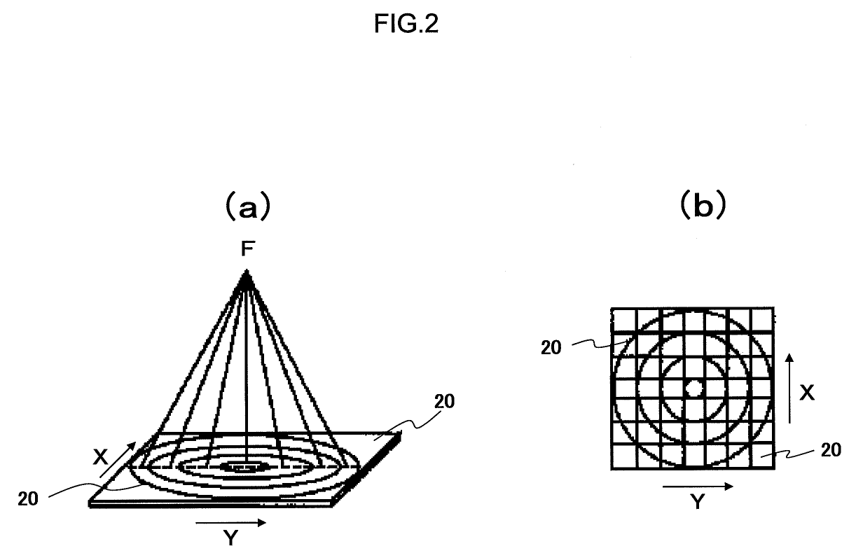

Next, basic concept of the Fresnel binding focus technique will be described using FIG. 2. FIG. 2 is a pattern diagram showing the basic concept of the Fresnel binding focus technique, indicating the pattern diagram wherein the ultrasonic beams of transmission/reception are formed on the central axis of the selected transducer bundles in the 2-dimensional array ultrasonic probe 10.

The respective transducers of 2-dimensional array transducers 20 are bundled by the switch control unit 22 in a circular or ring-pattern of the concentric circle, and selected so as to form a circular transducer bundle and a plurality of ring-shaped transducer bundles. Particularly the ring-shaped transducer bundles are formed by binding the transducers exist in the region intervened by two concentric circles having different radii (that is, the region within the same distance range from the center). And the plurality of transducers bundled in the same transducer group are transmitted/received at the same time. A plurality of line segment bundles extending from a point “F” to a transducer plane in FIG. 2(a) indicates the condition that the ultrasonic waves eradiated from the respective transducer bundles are bound in the point “F” (the focus point), and that the ultrasonic waves reflected from the point “F” are heading to the respective transducer bundles. In FIG. 2(b), 2-dimensional array transducers are set, for example, as 7×7 each in X-direction and Y-direction which are orthogonal to each other, and indicated by total of 49 transducers.

The 2-dimensional array transducers for actual diagnosis are arranged by, for example, in vertical and horizontal directions about 64×64=4096 transducers. Therefore, the transducer bundles to be selected in a ring pattern are finer, and a number of ring-shaped transducer bundles having different radii are to be formed.

As for the distance from the point “F” to the respective transducers for forming one ring, there is a slight difference between the distance to the transducers positioned in the inner side of the ring and the distance to the transducers positioned in the outer side of the ring. Therefore, it is preferable to set the upper limit in the difference between the radii of inner periphery and outer periphery of the ring in order to cause the transducers positioned in the inner periphery side and the outer periphery side of the ring to transmit/receive at the same time.

### FIRST EMBODIMENT

The first embodiment of the present invention will now be described. The characteristics of the present embodiment are as follows. The plurality of transducers bound as one transducers bundle transmit/receive ultrasonic waves at the same time in the same frequency and the same phase without the different delay times. Further, groups of transducer bundles are formed by dividing a plurality of transducer bundles corresponding to the frequency and the position of the focus points. The plurality of transducer bundles that belong to the same group transmit/receive in the same frequency. Also, at least two transducer bundles from the plurality of transducer bundles that belong to the same group transmits/receives ultrasonic waves in different phases. As for the segmentation of the plurality of transducer bundles, more focus points at different positions can be set by preferably assembling a pair of adjacent transducer bundles having different phases. However, in the case of reducing the number of focus points, more than two transducer bundles may be collected up to be set as one group. Also, it may be set so that different groups can have the same focus point. Hereinafter, the present embodiment will be described in concrete form.

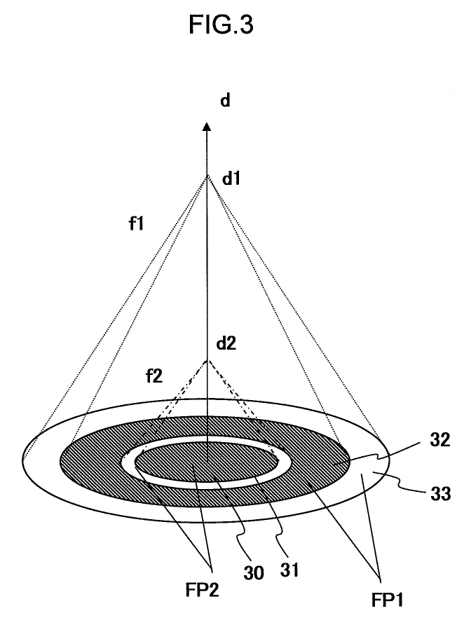

Features of the present embodiment will be described using FIG. 3. FIG. 3 shows the condition that each transducer of the 2-dimensional array transducers is bundled using Fresnel binding by the switch control unit 22, that is the condition that each transducer is selected and bundled so as to be in a circle or ring pattern of the concentric circle as a transducer bundle. Also, FIG. 3 shows the condition that the ultrasonic waves are transmitted from the respective transducer bundles that are bound by the Fresnel binding so that they are focused in two points of d1 and d2, the ultrasonic waves reflected from the two points of d1 and d2 are forwarded to the respective transducer bundles, and the respective transducer bundles receive them so that they will be focused in the two points of d1 and d2.

(Transmission Using the Fresnel Binding)

First, transmission using the Fresnel binding will be described. The switch control unit 22 and the transmitting unit 12 form a Fresnel bundle FP2 by raising transmission frequency for focusing the ultrasonic waves to “d2” in a shallow portion in the depth direction (“d”-direction), and form the Fresnel bundle FP1 by lowering transmission frequency for focusing ultrasonic waves to the “d1” in a deep portion in the depth direction (“d”-direction). In other words, since attenuation of high-frequency ultrasonic waves increases as they reach a deeper portion, the switch control unit 22 and the transmitting unit 12 form the Fresnel binding which uses the ultrasonic waves of lower frequency as focusing to the deeper portion, compared to the frequency in a shallow portion. Then the transmission will be performed by the Fresnel bundle FP1 and the Fresnel bundle FP2 simultaneously.

First, the case of focusing ultrasonic waves to “d2” in a shallow portion by the Fresnel bundle FP2 will be described. The switch control unit 22 selects a circular transducer bundle 20 in the central portion and a ring-shaped transducer bundle 31 which is adjacent to the outer side of the circular transducer bundle 30, and sets them as one group. Then the transmitting unit 12 performs focusing by ultrasonic waves of high frequency to a shallow portion using the selected one group of transducer bundles 30 and 31. For example, in the case of focusing ultrasonic waves to the depth of d2=10 mm, the transmitting unit 12 transmits the respective ultrasonic waves simultaneously by the frequency of f2=10 MHz using a group of the circular transducer bundle 30 and the ring-shaped transducer bundle 31. On this occasion, transmitting unit 12 makes the phases of the transducers for performing the Fresnel binding in the circular transducer bundle 30 the same upon transmission, and differentiates the phases of the transducers for performing the Fresnel binding in the ring-shaped transducer bundle 31 only by 180° (π) from the transmitting ultrasonic waves of the circular transducer bundle 30 upon transmission (hereinafter simply referred to as “antiphase”). In this way, as for the binding of transducers, the 2-dimensional array transducers are bound using the Fresnel binding focus technique (for example, Patent Document 2) wherein distance between the focus point and the transducer are standardized in increments of (transmitting signal wavelength/2) unit. The feature related to the present invention is that the phase of ultrasonic waves in adjacent transducer bundles of the same group are differentiated from each other upon transmission. By doing so, the respective ultrasonic waves that are transmitted simultaneously from each of the transducer bundles in the same group can be focused at a desired focus point, without appending specific focus data for each transducer which forms the 2-dimensional array transducers.

As for the plurality of transducer bundles of the same group to be transmitted at the same frequency, the width in the radial direction of the transducer bundle in the end side is to be made narrower than the width in the radial direction of the transducer bundle in the central portion side. Concretely, the width in the radial direction (that is the radius) of the ring-shaped transducer bundle 31 on the end-portion side is to be made narrower than the width in the radial direction of the circular transducer bundle 30 on the central portion side. In other words, the closer the ring-shaped transducer bundle is positioned to the end-portion of the probe, the narrower the width in the radial direction is to be made. Since the distance difference from the transducer to the focus point gets longer between the adjacent transducers as the position of the transducer gets closer to the end-portion side of the probe, it is necessary to make the width in radial direction (that is the ring width) of the ring-shaped transducer bundle narrower as the position of the transducer bundle gets closer to the end-portion of the probe in order to make the distance difference among the transducer bundles about the same. Describing from a different perspective, as for the plurality of transducer bundles of the same group for transmitting in the same frequency, the number of the transducers to be bound in the transducer bundle on the end side is to be made less than the number of transducers to be bound in the transducer bundle on the central portion side. Concretely, the number of transducers to be bound in the ring-shaped transducers bundle 31 on the end-portion side is to be made less than the number of transducers to be bound in the circular transducers bundle 30 on the central-portion side. If the frequency difference between the adjacent groups is minimal, the same above-mentioned feature can be applied also to the transducers belonging to the adjacent group thereof. That is, the closer the position of the ring-shaped transducer bundle gets to the end-portion of the probe, the narrower the width in the radial direction becomes and the fewer the number of transducers to be bound in the transducers bundle gets.

Next, the case of focusing ultrasonic waves to “d1” in a deep portion by the Fresnel binding FP1 will be described. The switch control unit 22 selects the ring-shaped transducer bundle 32 adjacent to the outer side of the ring-shaped transducer bundle 31 and the ring-shaped transducer bundle 33 adjacent to the outer side of the ring-shaped transducer bundle 32 and sets them as one group, at the same timing with the transmission of the above-mentioned circular transducer bundle 30 and the ring-shaped transducer bundle 31 as one group. In doing so, the switch control unit 22 makes the width in the radial direction of the ring-shaped transducer bundle 33 on the end-portion side narrower than the width in the radial direction of the ring-shaped transducer bundle 32 in the central-portion side (in other words, the number of transducers to be bound to the ring-shaped transducer bundle 32 on the end-portion side is to be made less than the number of transducers to be bound to the ring-shaped transducer bundle 32 on the central-portion side. Then the transmitting unit 12 transmits the ultrasonic waves at a lower frequency than the group of the circular transducer bundle 30 and the ring-shaped transducer bundle 31 in the inner side, using the group of the selected ring-shaped transducer bundles 32 and 33. For example, in the case of focusing ultrasonic waves to the deep portion in the depth of d1=50 mm, the transmitting unit 12 transmits the ultrasonic waves respectively at the frequency of f1=5 MHz using the group of the ring-shaped transducer bundle 32 and the ring-shaped transducer bundle 33. In doing so, the transmitting unit 12 transmits the ultrasonic waves by making the phase of the transducers to be bound to the ring-shaped transducer bundle 32 the same, and also by making the transducers to be bound to the ring-shaped transducer bundle 33 antiphase to the transmitting ultrasonic waves of the ring-shaped transducer bundle 32.

In this way, the signals from the Fresnel binding FP1 is focused to the depth “d1” at the frequency “f1”, and the signals from the Fresnel binding FP2 is focused to the depth “d2” at the frequency “f2” respectively. This makes it possible to form transmission beams focused to “d1” and “d2” by one transmission.

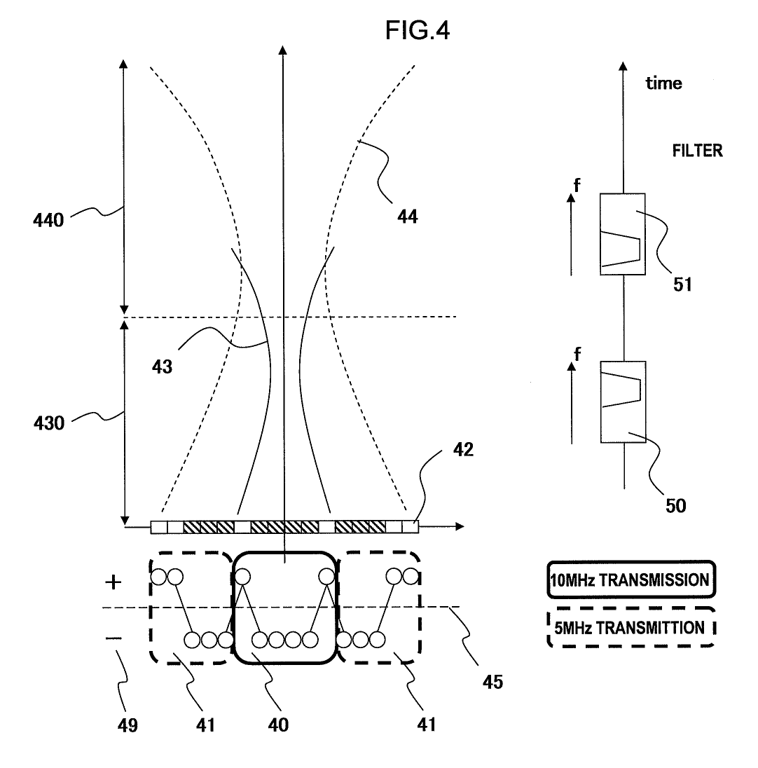

The above-mentioned transmission of ultrasonic waves will be described using FIG. 4. The same explanation is to be applied to the reception of ultrasonic waves to be described later. FIG. 4 shows a cross-section of the transducers 42 in the Y-axis direction passing through the center of the ring. While a transducer 42 a cross-section is represented as 16 elements for illustrative purposes, the actual cross-section is not limited thereto. Also, the region corresponding to the group of FP2 in FIG. 3 is set as a region 40, and the region corresponding to the group of FP1 is set as a region 41. The beam shape transmitted by the transducer 42 in the region 40 is indicated as “43”, and the beam shape transmitted by the transducer 42 in the region 41 is indicated as “44”.

A symbol “49” corresponding to the transducer 42 is a mark for representing the phase of the transmitting ultrasonic wave. The symbol in the case that the transducers are above a dashed line 45 is “+”, and the symbol in the case that the transducers are below a dashed line 45 is “−” which is an antiphase to the phase of the symbol “+”. Also, the symbol 49 corresponds to the transducer 42 on one-on-one level. The symbol 49 is denoted for an illustrative purpose, and is for illustrating that the transducer 42 having the same symbol is transmitted in the same phase, and the transducer 42 having different symbols is transmitted in antiphase.

For example, in the region 40, the circular transducer bundle corresponding to the four symbols in the central portion on the lower side transmits ultrasonic waves in a “−” phase, and the ring-shaped transducers corresponding to the one symbol on both edges of the upper side transmits ultrasonic waves in a “+” phase. Focusing is to be performed to a shallow portion at high frequency of 10 MHz, as one group formed by the plurality of transducers 42 corresponding to the phases of “+” and “−” in the region 40. In this way, the beam shape transmitted by the group of transducers 42 in the region 40 becomes as shown in “43”.

Also, in the region 41, the ring-shaped transducer bundle corresponding to the 3 symbols on the lower side transmits ultrasonic waves in a “−” phase, and the ring-shaped transducer bundle corresponding to the two symbols at the end portion adjacent thereto on the outer side transmits ultrasonic waves in a “+” phase. Focusing is performed to a deep portion at low frequency of 5 MHz, as one group formed by the plurality of transducers 42 corresponding to the phases of “+” and “−” in the region 41. In this way, the beam shape transmitted by the group of transducers 42 in the region 41 becomes as shown in “44”.

The transmissions to the above-mentioned focus 43 and focus 44 are performed simultaneously. This means that ultrasonic beams having a focus to two different depths can be formed in one transmission.

While the number of transducer bundles belonging to each group is set as two (that is a pair of transducer bundles having antiphase from each other) in the example shown in FIG. 4, the number of transducer bundles may be differentiated in at least two groups. Further, the number of transducer bundles in the same phase may be differentiated, without setting the transducer bundles in antiphase from each other as a pair. For example, the transducer bundles in the phase of “−”, “+” and “−” may be set as one group, or the transducer bundles in the phase of “+”, “−” and “+” may be set as one group.

Also, within the transducer bundles belonging to the same group, the width in radial direction of the transducers on the central portion side should be wider than the width in the radial direction of the transducers on the end-portion side (that is to make the number of transducers to be bound to the transducer bundles on the end-portion side to be less).

(Reception Using the Fresnel Binding)

Next, the reception using the Fresnel binding will be described.

Upon reception, the Fresnel binding pattern that are the same or different from the one upon transmission may be used. Hereinafter, the case of receiving ultrasonic waves using the same Fresnel binding pattern as the one upon transmission will be described in detail. In the same manner as transmission time, the transducer bundles are formed by binding the transducers, and a plurality of transducer bundles are divided so as to form a plurality of groups. Then the reception frequency of the plurality of transducers belonging to the same group is made the same to receive ultrasonic waves. Also, at least two transducer bundles within the plurality of transducers belonging to the same group receive ultrasonic waves in different phases. In this way, the receiving focus points are differentiated for every group. The concrete explanation of the above-mentioned reception will be described below.

The ultrasonic waves to be received include ultrasonic waves in different frequencies, such as 10 MHz and 5 MHz upon transmission. The control unit 18 receives the ultrasonic waves using the transducers 42 which are the same as the transmitted transducers 42. For example, the transducers 42 which receive the ultrasonic waves transmitted to the focus 43 are the transducers of the group in the region 40, and the transducers 42 which receive the ultrasonic waves transmitted to the focus 44 are the transducers of the group in the region 41.

(Filtering Process)

Next, the filtering process of the receiving signals will be described.

Since the focus 43 and the focus 44 have different depths, the times to receive the signals are also different. The focus 43 that is the ultrasonic waves in a shallow portion are received earlier, and the focus 44 that is the ultrasonic waves in a deep portion are received later. Thus, with respect to the ultrasonic waves obtained from the respective focus points, the signals from the respective focus points are selected by applying the filter corresponding to a high frequency or low frequency. Given this factor, the ultrasonic diagnostic apparatus of the present invention divides the receiving signals into several frequency bands by performing filtering process on the receiving signals using a plurality of filters having different passing bands placed in the reception phasing unit 13.

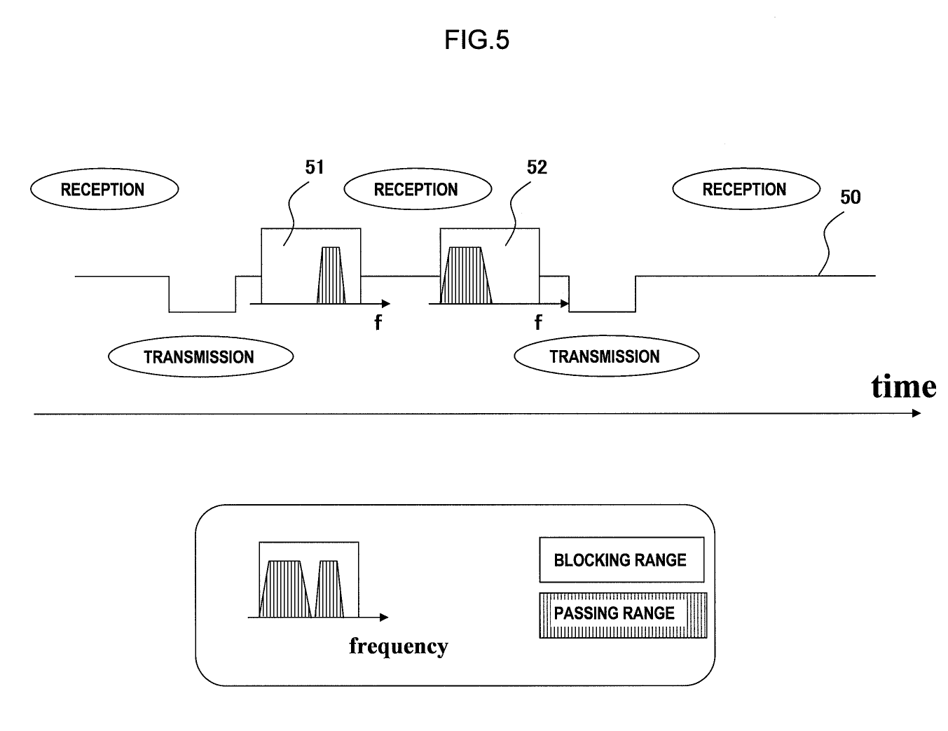

Here, the sequence of the transmission/reception timing will be described referring to FIG. 5. In a sequence 50 shown in FIG. 5, the rectangular period being convex on top represents the reception timing, and a rectangle period being convex underneath represents the transmission timing. Also, in a filter 51, the high-frequency receiving signals pass through, and the low-frequency receiving signals are blocked. In a filter 52, the low-frequency receiving signals pass through, and the high-frequency receiving signals are blocked. The corresponding relationship between timings of each filtering process and the depth of the focus point is illustrated in FIG. 4.

During the reception period, in the reception phasing unit 13, the reflected ultrasonic waves based on the high-frequency ultrasonic waves focused in a shallow region 430 are received in an earlier timing since the distance from the transducer to the focus position is closer. Therefore, in the earlier timing from the time that the transmission is switched to the reception, the high-frequency receiving signals are performed with the filtering process by the filter 51, and the low-frequency receiving signals are eliminated. Also, in the reception phasing unit 13, the reflected ultrasonic waves based on the low-frequency ultrasonic waves focused in a deep region 440 are received in a later timing since the distance from the transducer to the focus position is farther. Therefore, after passing of predetermined time from the filtering process by the filter 51, the low-frequency receiving signals are performed with the filtering process by the filter 52, and the high-frequency receiving signals are eliminated. When the filtering process by the filter 52 is completed and a predetermined time has passed, the reception timing is switched to the transmission timing, and the ultrasonic waves are transmitted. In accordance with such method, the transmission and the reception are alternately repeated.

As for the filter, a plurality of filters may be prepared for each frequency, or it may be the kind wherein the passing band of one filter can be dynamically changed from high frequency to low frequency in accordance with the reception timing. For example, the passing band can be changed by dynamically changing the coefficient of an FIR filter in accordance with the reception timing.

(Intensity Control of Ultrasonic Waves)

Next, the distance between the probe surface and the focus point, that is intensity control of transmitting ultrasonic waves in accordance with the depth will be described. Since an ultrasonic beam transmitted from the region on the end portion of a probe has tendency that the beam intensity gets attenuated compared to the ultrasonic beam transmitted from the central region of the probe, it is preferable that the beam intensity of ultrasonic beams in the end-portion region is made higher. Because the number of transducers to be used for transmission, distance of the focus point (depth) and extensity of focus which influence the beam intensity of ultrasonic waves in the living body are different between a deep portion and a shallow portion.

Concretely, the deeper the distance of the focus point is the weaker the beam intensity gets due to attenuation of the ultrasonic waves. Also, the fewer the number of transducers to be used for transmission of ultrasonic waves, the weaker the beam intensity gets. Also, the closer the region gets to the end-portion of the probe, the weaker the beam intensity gets due to extension of the focus of the transmitted ultrasonic beams. Given this factor, the transmitting unit 12 controls intensity of transmitting ultrasonic beams in accordance with the distance of the focus point (depth), the number of transducers and extensity of the focus. In other words, the intensity of ultrasonic beams on the end-portion side of the probe is made higher than the intensity of ultrasonic beams on the central-portion side so as to make the ultrasonic beams transmitted from the central-portion side of the probe and the ultrasonic beams transmitted from the end-portion of the probe to be practically in the same level at the focus point. Or, in the reception phasing unit 13, the receiving signals received on the end-portion side of the probe may be drastically amplified as compared to the receiving signals received in the central-portion of the probe. In order to drastically amplify the receiving signals, for example, gain of the preamplifier can be raised, or the value can be increased by multiplying the value by the coefficient after digitalizing the receiving signals.

(Frequency Control of Ultrasonic Waves)

Next, frequency control of transmitting ultrasonic waves in accordance with depth will be described. Distance resolution can be raised by variably transmitting the wavenumber in accordance with each depth. The wavenumber of the high-frequency ultrasonic waves to be transmitted toward a shallow portion in the group of the circular transducer bundle 30 and the ring-shaped transducer bundle 31 in the central-portion side of the probe 10 is to be set larger than the wavenumber of the low-frequency ultrasonic waves to be transmitted toward a deep portion in the group of the ring-shaped transducer bundle 32 and the ring-shaped transducer bundle 33 on the end-portion side of the probe. Conversely, the wavenumber of the low-frequency ultrasonic waves to be focused to a deep portion is to be set less compared to the wavenumber of the high-frequency ultrasonic waves to be focused to a shallow portion. For example, the wavenumber 97 of 4 waves is transmitted to a shallow portion, and the wavenumber 99 of two waves is transmitted to a deep portion. In the case of focusing to the shallow portion, there will be more portions that the waves are overlapped since the distance difference between the respective transducer bundles and the focus point is smaller. Thus the distance resolution can be increased by setting the larger wavenumber. Conversely, in the case of focusing to a deep portion, there will be fewer portions that the waves are overlapped since the distance difference between the respective transducer bundles and the focus point is larger. Thus the wavenumber need to be set fewer in order to raise the distance resolution.

As described above, in the present embodiment, the Fresnel binding is formed by setting higher frequency as the focus point is set in a shallower portion. Also, the deeper the portion gets for setting the focus point, the lower the frequency is to be set for forming the Fresnel binding. In other words, the most appropriate Fresnel binding pattern is determined corresponding to a plurality of sets of the focus point position in the depth direction and the frequency thereof. In this way, the ultrasonic beams which are focused to the plurality of focus points can be formed in one transmission/reception by applying the ultrasonic waves in accordance with the depth of the focus point for every group of transducer bundles. As a result, an image with no blur and with preferable SN can be obtained in a wide range from a shallow portion to a deep portion of the image without lowering the frame rate of the image or without using delay control for each transducer.

### SECOND EMBODIMENT

A second embodiment of the present embodiment will now be described. A difference from the first embodiment is that ultrasonic waves are transmitted/received to/from three focus points. Other points are the same as the first embodiments, thus the detailed explanation about the same points will be omitted. Hereinafter, the present embodiment will be described in concrete form.

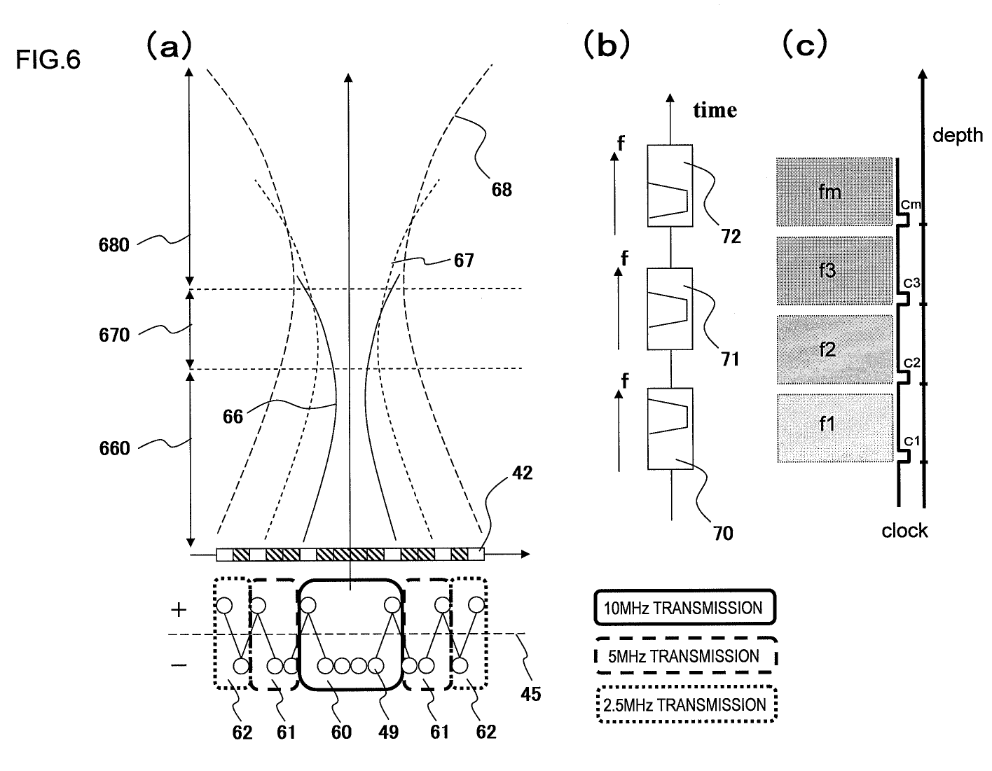

The feature of the present embodiment will be described referring to FIG. 6. The beam shape transmitted by the group of the transducers 42 in the region 60 is indicated by “66”, the beam shape transmitted by the group of the transducers 42 in the region 61 is indicated by “67”, and the beam shape transmitted by the group of transducers 42 in the region 62 is indicated by “68”.

As shown in FIG. 6(a), in the region 60, four symbols in the central part on the lower side are “−”, and one symbol on both ends in the upper side is “+”. In this way, the switch control unit 22 selects the transducers 42 from one region (group) wherein ultrasonic waves are transmitted/received in the same frequency so as to make the width of the transducer bundles in radial direction for transmitting from the end-portion side become narrower compared to the width of the transducer bundles in radial direction for transmitting from the central-portion side (that is to set the less number of transducers for binding to the transducer bundle in the end-portion side fewer). The transmitting unit 12 transmits the circular transducer bundle in which the transducers having the “−” symbol in the central portion side are bound by setting them as “−” phase, and transmits the ring-shaped transducer bundles in which the transducers having “+” phase in the end-portion side are bound and is adjacent to the circular transducer bundle by setting them as “+” phase. The bundles of the transducers 42 of “+” and “−” in the region 60 are transmitted as one group at the high frequency of 10 MHz, and focused to a shallow portion. In this way, the beam shape transmitted by the group of transducers 42 in the region 60 becomes “66”.

Also, in the region 61, two transducers in the inner side of the lower side are “−”, and one transducer in the end-portion side of the upper side is “+”. The switch control unit 22, in the same manner as the region 60, selects the transducers 42 so as to make the width in radial direction of the transducers to be transmitted from the end-portion side narrower compared to the width in radial direction of the transducers to be transmitted from the central-portion side. The transmitting unit 12 transmits the ring-shaped transducer bundle wherein the transducer 42 in the lower side are bound by setting them as “−” phase, and transmits the ring-shaped transducer bundle which is adjacent to the previously mentioned ring-shaped transducer bundle wherein the transducers 42 in the end-portion are bound by setting them as “+” phase. The bundle of the transducers 42 of “+” and “−” in the region 61 is transmitted as one group at the frequency of 5 MHz, and focused to an intermediate portion. In this way, the beam shape transmitted by the group of transducers 42 in the region 61 becomes “67”.

Also, in the region 62, one transducer in the inner side of the lower side is “−”, and one transducer in the end-portion side of the upper side is “+”. The transmitting unit 12 transmits the ring-shaped transducer bundle wherein the transducers 42 of the lower-side are bound by setting them as “−” phase, and transmits the transducer bundle which is adjacent to the previously mentioned ring-shaped transducer bundle wherein the transducers 42 of the end-portion side are bound by setting them as “+” phase. The bundle of the transducers 42 of “+” and “−” in the region 62 is transmitted as one group at the frequency of 2.5 MHz, and focused to a deep portion. In this way, the beam shape transmitted by the group of the transducers 42 in the region 62 becomes “68”.

The ultrasonic beams are transmitted to the above-described focus 66, focus 67 and focus 68 simultaneously. It means that ultrasonic beams having a focus to three different depths can be transmitted by one transmission.

As in the embodiment 1, while the switch control unit 22 has two transducer bundles belonging to the same group (which is a pair of transducer bundles having relationship being antiphase to each other) in the example shown in FIG. 6(a), the number of transducer bundles of at least one group may be different from the number of transducer bundles of other groups. Further, in the same group, the number of transducer bundles of the same phase may be differentiated without making them a pair of transducer bundles being antiphase to each other.

Also, since the depths among the focus 66, focus 67 and focus 68 are different upon reception, the time for receiving the reflected waves from each focus point will also be different. In the reception phasing unit 13, the focus 66 that is the ultrasonic waves in a shallow portion is received earlier, the focus 67 that is the ultrasonic waves in the intermediate portion are received next, and the focus 68 that is the ultrasonic waves in the deep portion is received at the last. As in the first embodiment, as shown in FIG. 6(b), the filter corresponding to each frequency is applied for each of the ultrasonic wave obtained from each focus point. A filter 70 for filtering the high-frequency of 10 MHz is applied to the focus 66 of a region 660, a filter 71 for filtering the intermediate-frequency of 5 MHz is applied to the focus 67 in a region 670 and a filter 72 for filtering the low-frequency of 2.5 MHz is applied to the focus 68 in a region 680.

As in the first embodiment, a plurality of filters may be prepared for each frequency, or the filter may be the one capable of dynamically changing the passing band of one filter from high frequency to low frequency in accordance with the reception timing.

Next, in the case of receiving ultrasonic waves using the Fresnel binding pattern different from the one upon transmission time will be described in detail.

Upon reception, the ultrasonic waves are received by setting the Fresnel binding pattern that is different from the one upon transmission time. For example, the switch control unit 22 differentiates the segments or group number of transducer bundles upon reception from those upon transmission. Then the reception phasing unit 13 differentiates the reception frequency of the respective groups or the reception phase of the respective transducer bundles upon reception from those upon transmission. Accordingly, the reception signals are phased by differentiating at least one out of the position of the focus point and the number of the focus points upon reception from the one upon transmission. As shown in FIG. 6(c), for example, after the transmitting unit 12 transmits the ultrasonic waves by focusing to one point, the switch control unit 22 and the reception phasing unit 13 receives them by setting a plurality of focus points. Concretely, upon receiving the receiving signals at clock C1, the reception will be performed using the group of transducer bundles in the region 60 which is in the central portion so as to be able to receive the ultrasonic waves reflected from a shallow portion. Then the ultrasonic waves are received using the group of the ring-shaped transducer bundles in the outer side region as the time elapses from clock C2 to clock C3. Lastly, upon reception of the receiving signals at clock Cm, the ultrasonic waves are received using the group of ring-shaped transducer bundle in the region 62 which is positioned at the end-portion.

The switch control unit 22 and the reception phasing unit 13 set the phase for the group of transducer bundles and each transducer bundle in the group respectively so that the ultrasonic waves reflected from the respective focus points can be received by the phase in compliance with the depth. For example, in the case of the clock C1, the phase is set for each transducer bundle of the group of transducer bundles in the region 60. The group of transducer bundles in the region 60 is a pair of transducer bundles which receive the signals in the same phase or in antiphase to each other, and receives the signals using the group formed by the pair of transducer bundles to which the phase is set.

In this way, the switch control unit 22 sets the transducer bundle for forming the Fresnel binding in accordance with the focus point, and divides the plurality of transducers into groups. The reception phasing unit 13 sets the reception frequency for each group, and sets the same phase or the antiphase with respect to every transducer bundle in the group. Then it receives the signals corresponding to the group of the transducer bundles, the frequency of the group thereof and the phase for each transducer bundle in the group with respect to every focus point, while changing the focus points. In this way, it is possible to set a plurality of focus points upon receiving ultrasonic waves even when only one or a few focus points are set upon transmission of the ultrasonic waves.

While the above-described example receives the signals by setting more focus points compared to the number of focus points upon transmission, it may be other way around by setting less focus points compared to the number of focus points upon transmission. In this way, it is possible to differentiate the number of focus points upon transmission time from the one upon reception time. Or, it also is possible to differentiate the position of the focus point upon transmission time from the one upon reception time.

Also, as in the first embodiment, intensity control of ultrasonic waves and frequency control of ultrasonic waves are can be performed in accordance with depth of the focus point.

As for the intensity control of ultrasonic waves, it is controlled so as to make the transmitting ultrasonic beams or the receiving signals to be practically the same level regardless of the depth of the focus point. Concretely, in the transmitting unit 12, the intensity of the ultrasonic beams to be transmitted toward the focus point in an intermediate portion may be made stronger compared to the ultrasonic beams to be transmitted toward the focus point in a shallow portion, and the intensity of ultrasonic beams to be transmitted toward the focus point in a deep portion may be stronger compared to the ultrasonic beams to be transmitted toward the focus point in the intermediate portion. Or, in the reception phasing unit 13, the receiving signals from the focus point in an intermediate portion may be drastically amplified compared to the receiving signals from the focus point in a shallow portion, and the receiving signals from the focus point in a deep portion may be drastically amplified compared to the receiving signals from the focus point in the intermediate portion.

In the same manner, the wavenumber of the ultrasonic waves to be transmitted toward the focus point in an intermediate portion may be increased compared to the wavenumber of the ultrasonic waves to be transmitted toward the focus point in a deep portion, and the wavenumber of the ultrasonic waves to be transmitted toward the focus point in a shallow portion may be increased compared to the wavenumber of the ultrasonic waves to be transmitted toward the focus point in the intermediate portion.

As mentioned above, in accordance with the present embodiment, as in the first embodiment, an ultrasonic beam can be formed wherein the ultrasonic waves are focused to a plurality of focus points in one transmission/reception, even in the case of setting three focus points. As a result, it is possible to obtain an image with no blur and with preferable SN in a wide range from a shallow portion to a deep portion in the image without lowering the frame rage of the image and without using a delay control for each transducer.

(Dividing the Transducers into Groups)

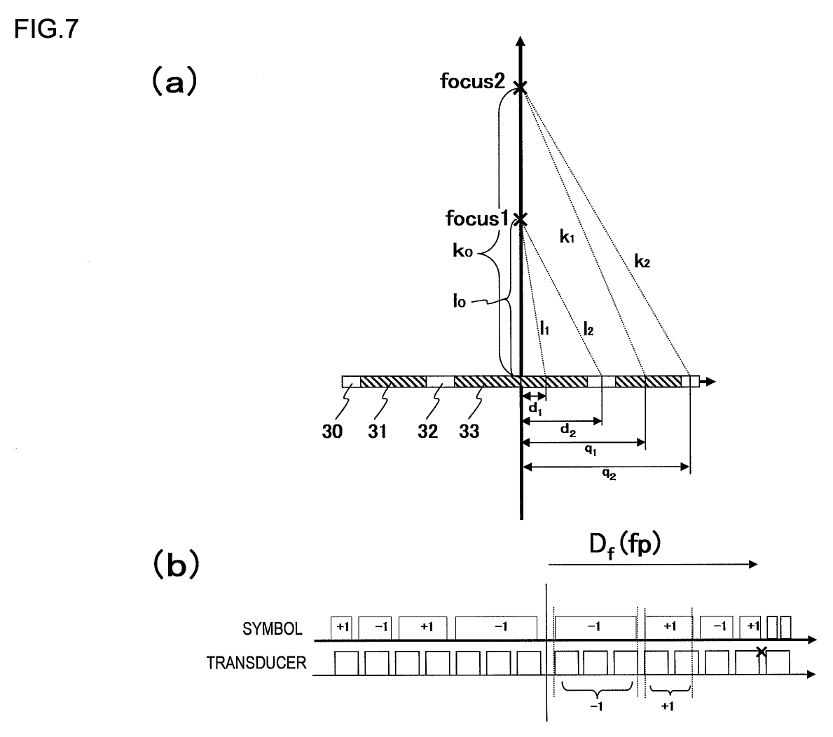

Next, basic principle on dividing the transducers into groups which is described above in relation to forming the transmission beams having a focus on a plurality of depths by one transmission and the reception thereof will be described using FIG. 7 and formulas. In the case of focusing ultrasonic waves to a focus point of “focus 1” in a shallow portion referring to FIG. 7(a), the distance “dn” from the center to the n-th transducer bundle is expressed as:

$$dn = \sqrt{{l}_{n}^{2} - {( {l}_{0} )}^{2}} . \qquad [ Formula 1 ]$$

Here, l₀ is the distance between the center of the surface of the transducer 42 and the focus point “focus 1”, and lₙ is the distance between the focus point “focus 1” and the n-th transducer bundle. And the following relational expression can be expressed between the adjacent transducer bundles:

lₙ−lₙ₋₁=λ_{fm}/2 [Formula 2]

Also, general formula of a waveform is expressed as:

λ_{fm}=v/fₘ [Formula 3]

And the upper limit of dₙ can be expressed as:

dₙ≦D_{f}. [Formula 4]

In this regard, however, D_{f} is the distance capable of forming the Fresnel binding for transmitting/receiving ultrasonic waves with respect to the “focus 1”. Relationship between the symbols and the transducers are shown in FIG. 7(b). In the right and left ends of the transducer, a plurality of symbols are allotted to one transducer where dₙ>D_{f}. In this way, the transducer to which two or more symbols are allotted is set as not for transmitting ultrasonic waves. In other words, the switch control unit 22 does not select the transducer to which two or more symbols are allotted, and selects the transducer bundle that is on the inner side of the unselected transducer to transmit/receive the ultrasonic waves. In the same manner, in the case of focusing the signals to a focus point “focus 2” in a deep portion, the distance from the center to the respective transducer bundles can be expressed as:

$${q}_{n} = \sqrt{{k}_{n}^{2} - {( {k}_{0} )}^{2}} . \qquad [ Formula 5 ]$$

Here, k₀ is the distance between the center of the surface of transducer 42 and the focus point “focus 2”, and kₙ is the distance between the focus point “focus 2” and the n-th transducer bundle. And the following relational expression can be expressed between the adjacent transducer bundles.

kₙ−kₙ₋₁=λ_{fp}/2 [Formula 6]

Also, general formula of a waveform can be expressed as:

λ_{fp}=v/fₚ. [Formula 7]

And the upper limit of qₙ can be expressed as:

qₙ≧Mₚ. [Formula 8]

In this regard, however, Mₚ is a distance capable of forming the Fresnel binding for transmitting/receiving ultrasonic waves with respect to the “focus 2”. Relationship between the symbols and the transducers are shown in FIG. 7(b). As D_{f} in the case of the focus point “focus 1”, in the right and left ends of the transducer, there are places where the symbol does not match with the transducer. Mₚ is the distance to the place thereof. In this way, the transducer to which two or more symbols are allotted is set as not for transmitting ultrasonic waves. In other words, the switch control unit 22 does not select the transducer to which two or more symbols are allotted, and selects the transducer bundle that is on the inner side of the unselected transducer to transmit/receive the ultrasonic waves.

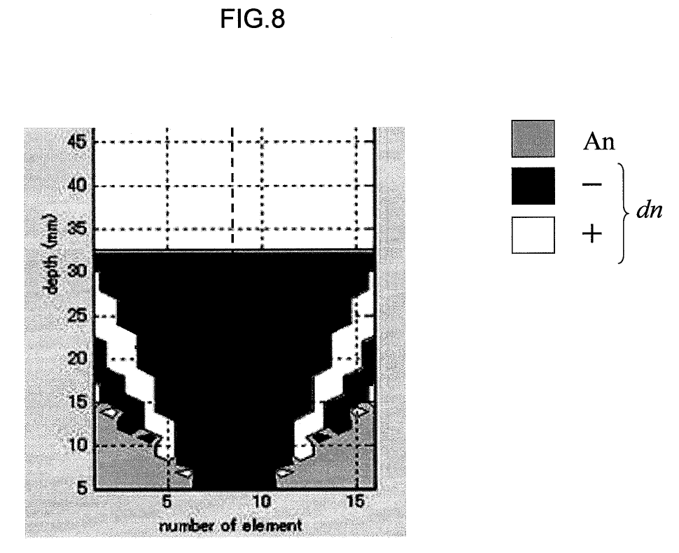

Here, the simulation on the relationship between the selection of transducers (appending symbols) and the depth using the above described formula is illustrated in FIG. 8. The simulation therein is performed using the circular transducer bundle 30 and the ring-shaped transducer bundle 31. The region indicated by black color is in “−” phase, and the transmitting signals to be transmitted by the circular transducer bundle 30 is conformed to the phase thereof. Also, the region indicated by white color is in “+” phase, and the transmitting signals to be transmitted by the ring-shaped transducer bundle 31 is conformed to the phase thereof. The region indicated by “An” is the region by which the transducer bundle is not selected.

For example, at the position in the vicinity of 10 mm depth, the circular transducer bundle 30 in the central portion is formed by seven transducers, and the ring-shaped transducer bundle 31 in the end-portion is formed by two transducers. Such defined transducer bundles are obtained based on the above-mentioned formula 1 and formula 2 respectively. On this occasion, since the upper limit of the distance capable of forming the Fresnel binding is surpassed in the “An” region, the transducer bundles in the region thereof is not to be used.

In the depth of 11 m ˜22 mm, the ring-shaped transducer bundle of “−” phase can be set on the further outer side of the ring-shaped transducer bundle 31. This ring-shaped transducer bundle of “−” phase can be utilized to execute the Fresnel binding focus in the same depth.

### THIRD EMBODIMENT

Next, the third embodiment of the present invention will be described. The difference from the first embodiment is that the transducers are configured having a plurality of microvibrational elements. Other than that is the same as the first embodiment and the detailed explanation on the same part will be omitted. Hereinafter, the present embodiment will be described in concrete form.

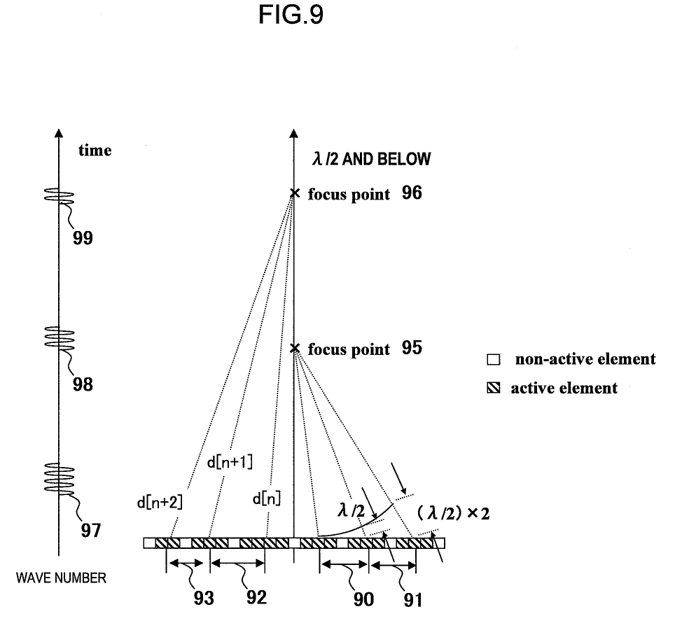

An example of a transducer configured having micro-vibrational elements will be described referring to FIG. 9. The transducer here is configured having, for example, a plurality of polygonal vibrational elements. The vibrational element is, for example, a nano-processed ultrasonic transducer of several micrometers. As vibrational element, for example, cMUT (Capacitive Micromachined Ultrasonic Transducer: IEEE Trans. Ultrasonic. Ferroelect. Freq. Contr. Vol45 pp. 678-690 May 1998, etc.) can be applied wherein the ultrasonic transmitting/receiving sensitivity that is electromechanical coupling coefficient varies in accordance with the intensity of bias voltage to be superimposed and applied to the driving signal provided from the transmitting unit 12. The cMUT is a nano-capacitance ultrasonic transducer manufactured by semiconductor nano-processing (for example, LPCVD: Low Pressure Chemical Vapor Deposition). In this regard, however, the nano-processed ultrasonic transducer can be applied without being limited to the cMUT. Such vibrational elements are aligned in equal or unequal intervals in major-axis direction “X” and the minor-axis direction “Y” to configure the transducer. In addition, other transducers are configured in the same manner.

Though in FIG. 9 an example for focusing to a deep portion is illustrated on the left side and an example for focusing to an intermediate portion is illustrated on the right side for illustrative purposes, an actual focus will be executed by the circular transducer bundles, the ring-shaped transducer bundles or the group thereof using both sides of the transducer.

In the case of focusing to a focus point 95 in an intermediate portion, ultrasonic waves are transmitted by binding 3 transducers each as a transducer bundle. The transducer bundle to be transmitted is set with a phase based on λ/2 (λ: wavelength of the ultrasonic wave) for each adjacent transducer. In other words, the transducer bundle within the range wherein the distance difference ΔL from the focus point 95 is λ/2 and below is transmitted by making the phase of the ultrasonic waves the same, and the transducer bundle is transmitted by rotating the phase of the ultrasonic wave by only π every time the distance difference ΔL from the focus point 95 surpasses λ/2. In the example shown in FIG. 9, to the respective three bundles of transducers on the left and the right side, the phase of α, α+π and α+2π (α is the initial phase) is set respectively from the central-portion side. In the same manner, in the case of focusing to the focus point 96 in a deep portion, four, three and two transducers from the central-portion side are bound as a transducer bundle, and the ultrasonic waves are transmitted by setting the phase of β, β+π and β+2π(is the initial phase) respectively. While the example of focusing by the group of three transducer bundles is illustrated in FIG. 9, the focusing may be performed by the group of two transducer bundles.

In this way, by normalizing the distance difference Δ L from the focus point by λ/2 unit, the kind of phase for transmitting/receiving signals are confined to two. More specifically, when the distance difference ΔL between the adjacent transducer bundles is half the wavelength or below, the phase of the ultrasonic waves for transmitting/receiving in the respective transducers thereof are made the same. Also, every time the distance difference ΔL between the adjacent transducer bundles surpasses half the wavelength, the phase of the ultrasonic waves for transmitting/receiving in the respective transducers thereof are made antiphase to each other through rotating only by π. In other words, as for the transducer bundles which satisfy the condition that “the phase difference φ(=ΔL/λ) based on the distance difference is 0≦φ<π”, the phase of the ultrasonic waves for transmitting/receiving are made the same. On the other hand, as for the transducer bundles which satisfy the condition that “the phase difference φ is π≦φ<2π”, the phase of the ultrasonic waves for transmitting/receiving is made antiphase to each other through rotating only by π with respect to the transducer bundles which satisfy the condition of “0≦φ<2π”.

Accordingly, the ultrasonic beams for focusing to the desired focus points among the plurality of transducer bundles are formed.

Also, from among the plurality of transducer elements, active element(s) and non-active element(s) are selected. The non-active elements here are the transducer element wherein the driving signals are not provided from the transmitting unit 12, or the transducer element wherein the direct-current bias is not applied. By adjusting the number for selecting the non-active elements, fine adjustment of the pitch among the groups can be performed. Also, by selecting the non-active transducer elements, it is possible to reduce cross talk generated among the groups. By such non-active transducer elements, a width 90 and a width 91 that are the pitch among the groups are set, the active transducer elements are set, and the transducer bundles are formed.

Also in the present embodiment, as in the first embodiment, it is possible to control the intensity and wavenumber of the ultrasonic waves in accordance with the depth of the focus point. In other words, the ultrasonic waves to be transmitted toward a focus point in a deep portion can be transmitted by making the intensity higher or making the wavenumber lower compared to the ultrasonic waves to be transmitted toward the focus point in a shallow portion.

As described above, in accordance with the present embodiment, freedom is increased with respect to the ultrasonic wavelength of the transducer pitch by using the transducers formed by the groups of microvibrational elements, whereby also increasing the freedom for combining the microvibrational elements to form the transducers. In other words, since the size of transducers can be variable with respect to the wavelength and can avoid the case that one transducer is allotted with a plurality of symbols as shown in FIG. 7(b), all of the transducers can be utilized effectively. As a result, intensity of the beams to be formed or azimuth resolution are improved.

### FOURTH EMBODIMENT

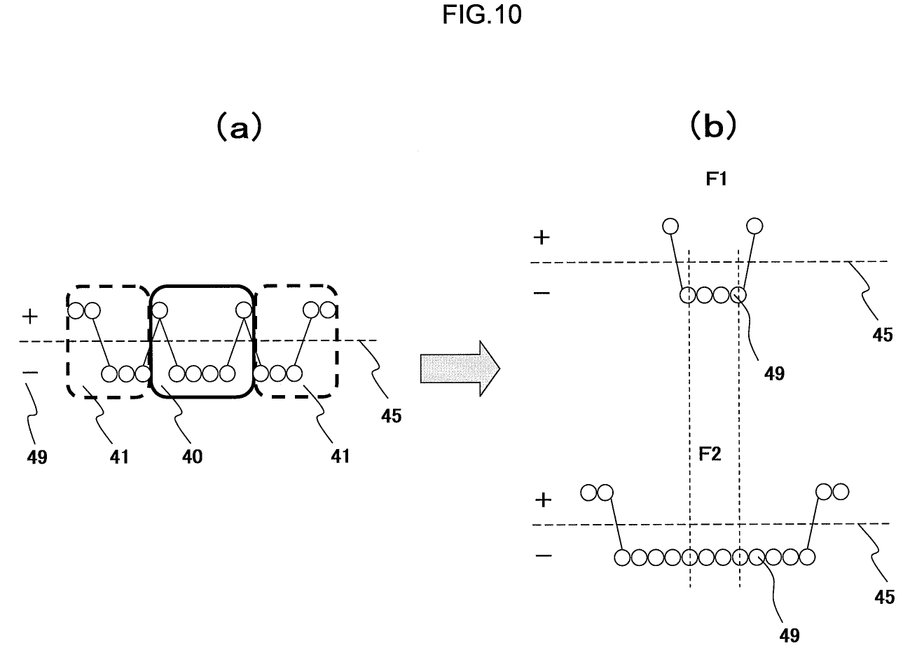

Next, the fourth embodiment of the present embodiment will be described referring to FIG. 10. While the transmitting ultrasonic beams are transmitted wherein ultrasonic waves are focused to “d1” and “d2” simultaneously by one transmission using the pattern of symbols shown in FIG. 10(a) in the above-described embodiments, they are transmitted by adding two transmitting signals created by varying the pattern of symbols in the present embodiment.

The transmitting unit 12, in the first pattern of symbols for focusing to a shallow portion, forms the transmitting signals using the symbols shown in the upper part of FIG. 10(b). Concretely, assuming the group formed by transducers 49 positioned at the central portion, the phase of the transducer bundle for bounding four transducers in the central portion on the lower side is set as “−”, and the phase of the transducer bundle for bounding one transducer each on both ends in the upper side is set as “+”. There is no symbol on the outer side of the above-mentioned both ends. That is, the transducers on the outer side than both ends thereof are not to be used. On the other hand, the transmitting unit 12 forms the transmitting signal using the symbols shown in the lower part of FIG. 10(b) in the second pattern of symbols for focusing to a deep portion. Concretely, assuming the group formed by further more transducers 49 positioned at the central portion, the phase of the transducer bundle for bounding 12 transducers in the central portion of the lower side is set as “−”, and the phase of the transducer bundle for bounding two transducers each on both ends in the upper side is set as “+”. The transmitting unit 12 adds the two transmitting signals created respectively as above, and forms the adding transmitting signals. By such adding transmitting signals, the transmitting beams having a focus in a different plurality of depths can be formed in one transmission.

The setting described in the present embodiment can be performed also upon reception, and it also is possible to set the phase of the respective transducer bundles so as to have a focus in a plurality of different depths in one reception.

As described above, in accordance with the present embodiment, appending symbols to each transducer bundle for each transmission/reception is not necessary since the waveform signals are created considering the focus point in advance.

### FIFTH EMBODIMENT

Next, the fifth embodiment of the present invention will be described. While the pattern for executing the Fresnel binding using 2-dimensional array transducers is described in the previous embodiment, the principle of the Fresnel binding related to the present invention is applied to one-dimensional array transducers in the fifth embodiment. That is to apply the pattern illustrated in the respective first ˜fourth embodiments to one-dimensional array transducers. For example, the allocation of the symbols (phases) shown in FIG. 4 and FIGS. 6˜9 are applied to one-dimensional array transducers.

Concretely, the cross-section in FIG. 4 is regarded as a one-dimensional transducer, and the transducers included in the same distance range from a predetermined one point (for example, the center) is selected and bound. In other words, the phase symbol “−” and the phase symbol “+” are appended to the respective one-dimensional array transducers 42, and the transducers to which the same phase is appended are bound to make a transducer bundle. Then a plurality of groups for transmitting/receiving ultrasonic waves by combining a pair of adjacent transducer bundles of different phases are set, and transmission/reception of ultrasonic waves is executed using the plurality of groups. The detailed description on the other aspects of the present embodiment will be omitted here to avoid repetition of the description above. In this way, also in one-dimensional array transducers, it is possible to form an ultrasonic beam capable of focusing ultrasonic waves to focus points in a plurality of different depths in one transmission, and to obtain the receiving signals that are phased so as to focus to the focus points thereof upon reception.

In accordance with the above-described present embodiment of one-dimensional array transducers, as in the case of the previously mentioned 2-dimensional array transducers, it is possible to reduce focus data and to form the ultrasonic beams that are focused in different frequencies for each of the plurality of different focus points in one transmission/reception. As a result, also in the case of one-dimensional array transducers, it is possible to obtain images with no blur and with preferable SN in a wide range from a shallow portion to a deep portion without lowering frame rate of the image nor using delay control for every transducer.

While the above respective embodiments described the pattern for forming the focus in two different depths and three different depths respectively upon transmission/reception of ultrasonic waves, no limitations are intended to the content disclosed in the above-described embodiments, and the intension is to cover all modifications, equivalents and alternatives falling within the spirit and scope of the invention. For example, a focus can be formed in four or more different depths by selecting more transducers in detail in the switch control unit 22. Also, while the pattern for differentiating the two kinds of phases which are “+” and “−” in the above-described respective embodiments, the different phases to be allotted does not have to be limited to two and may be three kinds or more (for example, 0, π/3 and 2π/3).

Also, while the pattern for focusing the group in the central-portion side to a shallow portion and the group in the end-portion side to a deep portion is described in the above respective embodiments, by contraries, the group in the central-portion side may be focused in a deep portion and the group in the end-portion side may be focused in a shallow portion.

Also, while the pattern for transmitting/receiving signals of the respective groups simultaneously is described in the above respective embodiments, the respective groups may transmitted/received signals independently for every focus point. For example, only groups in the central portion of a probe may transmit/receive ultrasonic waves toward/from the focus point in a shallow portion, then only groups in the end-portion of the probe may transmit/receive ultrasonic waves toward/from the focus point in a deep portion, to construct an image using the respective receiving signals. Or, as described in the Patent Document 1, one frame data may be obtained by transmitting/receiving signals toward/from the focus point in a shallow portion by only the groups in the central portion of the probe, then the next frame data may be obtained by transmitting/receiving signals toward/from the focus point in a deep portion by only the groups in the end-portion of the probe, and the operations thereof may be repeated alternately.

### BRIEF DESCRIPTION OF THE DIAGRAMS

FIG. 1 shows a block diagram for illustrating the general configuration of the present invention.

FIG. 2 is a pattern diagram showing the formation of an ultrasonic beam for transmission/reception in the central portion of the 2-dimensional array transducer.

FIG. 3 shows the feature of the first embodiment of the present invention.

FIG. 4 is shows a cross-section of a ring formed by 2-dimensional array transducers.

FIG. 5 is a sequence diagram of the transmission/reception timing.

FIG. 6 shows the feature of the second embodiment in the present invention.

FIG. 7 shows the region segmentation of a transducer.

FIG. 8 is a block diagram illustrating the general configuration of the present invention.

FIG. 9 shows the feature of the third embodiment in the present invention.

FIG. 10 shows the feature of the fourth embodiment in the present invention.

### DESCRIPTION OF THE SYMBOLS

10 . . . ultrasonic probe, 11 . . . transducer selecting data unit, 12 . . . transmitting unit, 13 . . . reception phasing unit, 14 . . . transmission/reception separating circuit, 15 . . . signal processing unit, 16 . . . scan converter, 17 . . . monitor, 18 . . . control unit, 23 . . . input unit

1. An ultrasonic diagnostic apparatus comprising:
an ultrasonic probe having a plurality of transducers for transmitting/receiving ultrasonic waves;
transmitting/receiving means for transmitting/receiving ultrasonic waves by focusing to each of a plurality of focus points; and
image acquiring means for acquiring an ultrasonic image using the receiving signals from the respective focus points,
characterized in further comprising transducer-group forming means for forming a group of transducers for each focus point, by selecting, from the plurality of transducers, a plurality of transducers which transmit/receive signals by the same transmitting/receiving frequency,
wherein the transmitting/receiving means corresponds to the position of the respective focus points, and transmits/receives the ultrasonic waves by differentiating the transmitting/receiving phases of at least two transducers which belong to the same group.

2. The ultrasonic diagnostic apparatus according to claim 1, wherein the transmitting/receiving means transmits/receives ultrasonic waves by each group independently with respect to every focus point.

3. The ultrasonic diagnostic apparatus according to claim 1, wherein the transmitting/receiving means transmits/receives ultrasonic waves of the respective groups simultaneously.

4. The ultrasonic diagnostic apparatus according to claim 1, wherein the transmitting/receiving means differentiates the transmitting/receiving frequencies in at least two groups.

5. The ultrasonic diagnostic apparatus according to claim 1, wherein the transmitting/receiving means makes the transmitting/receiving frequency of the group corresponding to the focus point positioned in a shallow portion which is closer to the transmitting/receiving surface of the ultrasonic probe higher than the transmitting/receiving frequency of the group corresponding to the focus point positioned in a deep portion which is farther from the transmitting/receiving surface of the ultrasonic probe.

6. The ultrasonic diagnostic apparatus according to claim 1, wherein the transmitting/receiving means makes the transmitting/receiving frequency of the group in the central-portion side of the transmitting/receiving surface of the ultrasonic probe higher than the transmitting/receiving frequency of the group in the end-portion side.

7. The ultrasonic diagnostic apparatus according to claim 1, wherein the transmitting/receiving means makes the intensity of the ultrasonic signals to be transmitted toward the focus point positioned in a deep portion which is farther from the transmitting/receiving surface of the ultrasonic probe higher than the intensity of the ultrasonic signals to be transmitted toward the focus point positioned in a shallow portion which is closer to the transmitting/receiving surface of the ultrasonic probe.

8. The ultrasonic diagnostic apparatus according to claim 1, wherein the transmitting/receiving means sets the focus point of the group positioned at the central-portion side of the transmitting/receiving surface of the ultrasonic probe to a shallow portion which is closer to the transmitting/receiving surface thereof, and sets the focus point of the group positioned at the end-portion side to a deep portion which is farther from the transmitting/receiving surface thereof.

9. The ultrasonic diagnostic apparatus according to claim 1, wherein the transmitting/receiving means receives the signals from a shallow portion using the group positioned at the central-portion side of the transmitting/receiving surface of the ultrasonic probe, and receives the signals from a deep portion using the group positioned at the end-portion side.

10. The ultrasonic diagnostic apparatus according to claim 1, wherein the transmitting/receiving means differentiates the number or position of the focus point upon transmission from the one upon reception.

11. The ultrasonic diagnostic apparatus according to claim 1, wherein the transmitting/receiving means makes the number or the position of the focus point upon transmission the same as the one upon reception.

12. The ultrasonic diagnostic apparatus according to claim 1, wherein the transmitting/receiving means differentiates the transmitting/receiving phases of at least two transducers which belong to the same group by π.

13. The ultrasonic diagnostic apparatus according to claim 1, wherein the transmitting/receiving means makes the difference number of the transmitting/receiving phases and the value of the respective transmitting/receiving phases the same in at least two groups.

14. The ultrasonic diagnostic apparatus according to claim 1, wherein the transmitting/receiving means applies a high-frequency pass filter for blocking the passing of low-frequency receiving signals by passing high-frequency receiving signals with respect to the receiving signals from a shallow portion, and a low-frequency pass filter for blocking the passing of high-frequency receiving signals by passing low-frequency receiving signals with respect to the receiving signals from a deep portion respectively.

15. The ultrasonic diagnostic apparatus according to claim 1, wherein the transmitting/receiving means makes the wavenumber of the ultrasonic waves to be transmitted using the group positioned at an end-portion side of the transmitting/receiving surface of the ultrasonic probe fewer than the wavenumber of the ultrasonic waves to be transmitted using the group positioned at a central-portion side.

16. The ultrasonic diagnostic apparatus according to claim 1, wherein:
the plurality of transducers are one-dimensional array transducers, and
the transducer group forming means selects the transducers included in the same distance range from a predetermined point and makes them as one group.

17. The ultrasonic diagnostic apparatus according to claim 1, wherein:
the plurality of transducers are 2-dimensional array transducers, and
the transducer group forming means selects the transducers included in the same distance range from a predetermined point and forms a circular or ring-shaped group of the concentric circle.

18. The ultrasonic diagnostic apparatus according to claim 1, wherein the transducers are cMUT transducers.

19. The ultrasonic diagnostic apparatus according to claim 16 or 17, wherein the transmitting/receiving means transmits/receives ultrasonic waves by adding the combination pattern and phase pattern of the transducers for focusing to a shallow portion, and the combination pattern and phase pattern of the transducers for focusing to a deep portion.

20. An ultrasonic diagnostic method comprising:
a transmitting/receiving step for transmitting/receiving ultrasonic waves by focusing to each of a plurality of focus points using an ultrasonic probe configured having a plurality of transducers; and
an image acquiring step for acquiring an ultrasonic image using the receiving signals from the respective focus points,
characterized in further comprising a step for forming a group of transducers by selecting, from the plurality of transducers, a plurality of transducers for transmitting/receiving in the same transmitting/receiving frequency with respect to every focus point,
wherein the transmitting/receiving step corresponds to the position of the respective focus points, and transmits/receives ultrasonic waves by differentiating the transmitting/receiving phases of at least two transducers which belong to the same group.
